(19) United States
(12) Patent Application Publication
(10) Pub. No.: US 2023/0040551 A1
(43) Pub. Date: Feb. 9, 2023

(54) IRRADIATION APPARATUS

(71) Applicant: JK-HOLDING GMBH, Windhagen (DE)

(72) Inventor: Jürgen GERSTENMEIER, Neuwied (DE)

(21) Appl. No.: 17/759,111

(22) PCT Filed: Jan. 22, 2021
(86) PCT No.: PCT/IB2021/050493

§ 371 Date: Jul. 20, 2022

(30) Foreign Application Priority Data
Jan. 22, 2020 (CH) 00071/20

(51) Int. Cl. A61N 5/06

(52) U.S. Cl. CPC A61N 5/0613

(57) ABSTRACT

The invention relates to an irradiation apparatus for irradiating an irradiation object with light, comprising at least one light source with an identification unit that contains light source-specific information; a frame, more particularly a reflective frame, at least one light source holder on which the at least one light source is arranged by form-fitting insertion in the light source holder; and a region which provides a detection field of a communications device; the identification unit being positioned on the at least one light source such that the identification unit, by form-fitting insertion in the light source holder, is within the detection field.

The present invention relates to a device and method for irradiating living beings with suitable light sources, each according to the preambles of the independent claims.

### TECHNICAL BACKGROUND

Devices for irradiating living beings, in particular the human body or parts of the human body, with optical radiation are known. These are used in the medical, cosmetic and/or therapeutic field. In the field of skin irradiation, emitting arrangements are used whose radiation produces a photobiological effect on an irradiated person, for example. The radiation hits the skin of a person, but depending on the specific wavelength it can penetrate into deeper regions of the body. The effect includes, for example, tanning of the skin, but also other physiological and psychological effects result from the radiation. The radiation includes the spectrum of ultraviolet (UV) radiation, visible (VIS) radiation and near infrared (nIR) radiation. The UV radiation has wavelengths in the spectrum between 100 nm and approx. 380 nm, the VIS radiation has wavelengths in the spectrum between approx. 380 nm and approx. 780 nm, the nIR radiation has wavelengths in the spectrum between about 780 nm and about 1400 nm. The spectra mentioned merge into one another. Depending on the application, the irradiation can be concentrated on a partial spectrum of the spectra mentioned. For this purpose, medical-cosmetic radiation-emitting arrangements can also be dedicated to individual wavelengths, for example the UV radiation that is generated by radiant tubes.

Devices for acting on the skin of a user are known from practice, such as those used in tanning salons, for example, in which a person to be irradiated can lie on a cover forming a resting surface or end surface for the purpose of tanning their skin through pigmentation, wherein an arrangement emitting UV radiation, usually with a plurality of radiant tubes, in particular fluorescent tubes, is arranged below the cover. Such tanning devices usually also have a further structural unit with further radiant tubes and a second cover, which can be pivoted together onto the person to be irradiated, so that the person can be tanned all around and evenly.

It goes without saying that it is of central importance that approved light sources are always used if they are used for the purpose of irradiating living beings, in particular the human body or parts of the human body. In particular, care must be taken to ensure that the light sources in an irradiation device always have a constant output and that they are replaced according to their service life. In case of a solarium, for example, a lifespan of 100 to 1000 hours is expected, depending on the type of manufacture and the type of use. There are measuring devices that can be used to determine the luminous intensity of light sources. However, this requires human resources.

The use of the right light sources is also of crucial importance for the success of the irradiation.

There is therefore a need for a device of the type mentioned at the outset, in particular a device and method for irradiating living beings with suitable light sources, in which the suitable light sources can be identified as simply and clearly as possible.

It is an object of the invention to specify an irradiation device for exposure to light, in which only suitable light sources are used, in particular with which damage to living beings or to the irradiation device can be avoided.

It is a further particular object of the present invention to provide such a device and a method which eliminates at least one disadvantage of the known art, in particular a simple and inexpensive device and method are to be provided.

### PRESENTATION OF THE INVENTION

According to the invention, the object is attained by a device having the features of the independent claims.

According to one aspect of the invention, an irradiation device for subjecting an irradiation object or a living being to light is specified. The device comprises at least one light source with an identification unit that contains light source-specific information. Furthermore, the device includes a frame, in particular a reflective frame, with at least one light source holder, on which the at least one light source is arranged. The light source is fastened by form-fitting insertion into the light source holder. A defined or predetermined region provides a detection field for a communication device. The identification unit can be positioned on at least one light source in such a way that the identification unit is located within the detection field due to the form-fitting insertion into the light source holder.

In a particular embodiment, the detection field extends at an angle from the frame, in particular essentially perpendicularly from the frame. As a result, particularly good communication between the identification unit and the communication device can be achieved. In particular, particularly good communication can be achieved between a specific identification unit and a specific communication device.

The frame is preferably metallic and defines an irradiation direction. The frame can be designed as a metal trough, so that a shielding function is provided.

In a particular embodiment of the present invention, the frame can influence the configuration of the detection field, in particular due to the material used, by virtue of its shielding properties.

In terms of the present invention, the frame designed as a metal trough can be considered as a reflective frame. Alternatively and/or additionally, the frame can be designed with a reflective layer. In the context of the invention, reflective is to be understood primarily with regard to the electromagnetic radiation emitted by the light source, in particular the reflection of light. The reflection effect can also be achieved with other means in addition to or as an alternative to the metallic configuration and/or coating. For example, faceted reflectors can be provided whose reflective surface is faceted for improved light reflection, for example in a pattern with diamond-shaped surfaces. The frame is particularly preferably made of faceted, aluminum-coated plastic.

In a special embodiment, the region is designed as a recess in the frame, preferably also as a milled-out region. If the defined region is non-metallic and preferably consists of a plastic, then an improvement in the inductive coupling between the identification unit and the communication device can be achieved.

The identification unit contains a specific code so that each light source can be uniquely identified. It is then possible to make a safe decision about the operation of the light source.

In a special embodiment, the irradiation device comprises a plurality of fluorescent tubes as a light source. These fluorescent tubes have a longitudinal extension and at least one, usually two end elements. For the purposes of the present invention, fluorescent tubes can be understood to mean commercially available gas discharge tubes in which a glow discharge is ignited between electrodes by applying a voltage and which then light up. However, the application of the teaching according to the invention also extends to LED light fixtures as light sources which, for example, can also be used in tube form with the appropriate plug-in connection, for example as a replacement for gas discharge tubes.

In a special embodiment, a plurality of light sources designed as fluorescent tubes are arranged in parallel as an irradiation field. Each of these fluorescent tubes can have an individual identification unit.

In a further special embodiment, the identification unit is provided at a specific point along the length of the light source. Particularly preferred for all light sources in the same position. Likewise, additionally or alternatively, particularly preferably substantially in the center, in particular in the center of a light source element of the light source that extends longitudinally in the form of a tube.

In a particular embodiment, the identification unit is attached to the outer circumference of a light source element of the light source that extends longitudinally and in the form of a tube. Alternatively, the identification unit is attached to the inner circumference of a light source element of the light source that extends longitudinally in the form of a tube.

In a particular embodiment, the identification unit is materially connected to the light source, in particular on the outer circumference or on the inner circumference. For this purpose, the identification unit can be accommodated in a glass mold and/or in a plastic casing, for example, and this can be glued or welded onto the outer circumference or inner circumference. The identification unit is particularly preferably connected to the light source in a tamper-proof manner. This can be achieved, for example, by manipulation of the identification unit leading to irreparable damage to it. For example, the identification unit can be in the form of an RFID transponder, the antenna of which extends flatly on the outer circumference of a light source element of the light source that extends longitudinally and in the form of a tube, so that an attempt to detach it leads to the antenna breaking or buckling, thus rendering the transponder ineffective.

In a further special embodiment, the identification unit is accommodated in a chamber formed in one piece on the light source.

In a particular embodiment, the identification unit is accommodated and/or designed to be thermally stable. This can be accomplished, for example, by designing the identification unit to function in an elevated (relative to room temperature) temperature range without restriction. In a very specific embodiment, the identification unit is housed in a thermally stable manner by being housed in an essentially thermally insulated space in or on the light source. This can be done, for example, in the form of a glass mold in which the identification unit is embedded and which has an insulating layer, for example an air layer. Alternatively and/or in addition, the identification unit can be designed to be thermally stable in that it essentially consists of materials which, without deforming or otherwise changing their properties, can be used in a temperature range of preferably up to 500 degrees Celsius, preferably up to 200 degrees Celsius. For the purposes of the present invention, a glass mold is to be understood as a uniformly designed, hermetically sealed molded glass body that includes an inner cavity. The identification unit would be accommodated in this cavity.

In a special embodiment, the identification unit is designed as an RF identification unit. The RF identification unit is particularly preferably designed in such a way that information can be read and/or written. This can be achieved with a memory on the RF identification unit that can be written to at least once. In particular, the memory can be written to repeatedly.

In a particular embodiment, the light source comprises at least one, preferably two, end elements, which are designed to interact with a light source base of the irradiation device. This active connection preferably has a plug-in connection, in which at least one plug-in extension can be brought from an end element into electrical connection with a socket of the corresponding light source base. In a particular embodiment, the light source comprises a plurality of plug-in extensions which, in addition to the plug-in connection for electrical connectivity with corresponding sockets of the base, enable a form fit in the installed state. In a particularly preferred embodiment, in which the light sources comprise light source elements extending longitudinally in the form of tubes, the plug-in extensions are brought into a form-fitting hold by rotating the light source about the longitudinal axis. In this embodiment, for example, first guide grooves, in particular tangential to the tube diameter, can be provided on the bases, which receive the plug-in extensions and then transfer them into second guide grooves, which in particular are radial to the tube diameter. A rotation of the light source around the longitudinal axis would correspond to a rotation along the guide grooves running radially to the tube diameter. In the tangential direction, such a light source would be blocked in its movement by a form fit.

In a particularly preferred embodiment, the identification unit is positioned on the light source in such a way that, in the installed state, it is positioned within the detection field due to the form-fitting insertion into the light source holder.

In a particularly preferred embodiment, the identification unit is positioned on the light source in such a way that after rotating the light source about the longitudinal axis by an angle of between 35 and 160 degrees, preferably between 45 and 125 degrees, it comes to rest within the detection field. In a particular embodiment, the identification unit is positioned on the light source in such a way that it comes to rest at right angles to the communication device after the light source has been rotated. In terms of the present invention, a rotation about the longitudinal axis can be considered, for example, in case of a positioning of a light source with two pins. This light source can be provided with corresponding insertion compartments or grooves for accommodation in a light source holder. In this embodiment, a rotation about the longitudinal axis of the light source in both directions, ie by + or −45 to 90 degrees, for example, would result in the light source locking in the light source holder and/or contacting the pins with the power supply. In this specific example, the identification unit would then be offset with respect to the orientation of the pins by an angle of between + or −45 to 90 degrees, so that the identification unit comes to rest in the detection field when rotated.

A further advantage of the present invention can be that, for example, in the case of light sources with a defined direction of irradiation, incorrect installation is impossible. Such a light source could, for example, have a reflective surface that only allows irradiation in one direction, by accumulation. Incorrect mounting, ie mounting with the reflecting side pointing into a radiation region and thus preventing radiation into this region, would not be possible since the identification unit would not be located in the detection field.

In a particular embodiment, the detection field extends at an angle of between 90 and 135 degrees from a normal of the communication unit.

In a particularly preferred embodiment, the identification unit is positioned on the light source in such a way that, after rotating the light source about the longitudinal axis, it comes to rest at a distance of 2 mm or less or between approximately 1 and 2 mm from the communication device.

It is advantageous if the distance between the identification unit and the communication device is greater than the distance between the communication device and the defined region, since the communication device can then be placed directly in or on the defined region.

In a further particular embodiment, the distance between the identification unit and the communication device is between 1 and 2 mm or 3 and 9 mm, in particular approximately 6 mm, and the distance between the communication device and the frame is approximately half of this.

In the context of the present invention, a size indication with “approx.” is to be understood as encompassing a natural, manufacturing-related variation usually within between 0.01 and 5%.

In a particular embodiment, a plurality of light sources are each equipped with an identification unit and a communication device is assigned to each identification unit, so that there is a unique assignment. Alternatively and/or in addition, a plurality of light sources can each be equipped with an identification unit, and the communication device can be designed for bulk detection, so that only one communication device is required. In particular, the communication device can be designed to recognize a plurality of identification units one after the other by keeping a registration log and logging received serial numbers. After one-off or repeated runs, there can then be a singulation of the individual identification units, for example. Additionally and/or alternatively, identification unit(s) and/or communication device(s) can be designed with anti-collision protocols. The communication device and/or plurality of communication devices is particularly preferably designed to localize the identification unit(s) using the respective detection field and/or plurality of detection fields. This can be done in a particular example by a protocol selected from the group consisting of: triangulation, probabilistic analysis, deterministic analysis, localization, e.g. based on previous calibration according to distance, direction, etc, frequency variation between the different identification units, etc.

In a special embodiment, the communication device includes a near-field transmitter/receiver. In particular, this can be designed to generate an antenna field in the detection region and to activate an identification unit located in the detection region. In this specific example, the identification unit would include a passive transponder without its own power supply.

In a particular embodiment, the communication device(s) is/are designed to be controllable. The controllability can be designed in such a way that a performance of the communication device can be modulated. In particular, a modulated performance can run as a search process in which the communication device(s) search for and/or recognize any identification unit(s) located in the detection region.

The communication device can be connected to an evaluation unit in order to process the information stored by the identification unit.

In a special embodiment, the communication device and/or the evaluation unit includes a storage medium. The storage medium can be provided, for example, as a local data storage device for storing data, or also alternatively or additionally as a wireless connection in particular to an external storage device. In a further special embodiment, the storage medium comprises a removable medium.

In a further special embodiment, the storage medium is designed to store a data packet that can be uniquely assigned to a light source. Particularly preferably, the data packet includes data selected from the group consisting of: specific coding for unique identification of the corresponding light source, mode and type of light source, date of commissioning and/or first detection of the light source, operating time of the light source, etc.

In a further special embodiment, the evaluation unit is designed to perform an automatic control of the light source based on the information stored on the storage medium and/or the information detected by the communication device. For example, a drop in the power of the light sources can be recorded and tracked. Furthermore, the evaluation unit can be designed to control the light source with regard to an optimized or legally required power output, for example an irradiation intensity. It can happen, for example, that regulatory requirements for the irradiation of living beings, in particular the human body or parts of the human body, with optical radiation in the medical, cosmetic and/or therapeutic sector are different from one another. The regulatory requirements, for example, can also differ from country to country. With the solution of the present invention it is possible to provide light sources and uniform irradiation devices which, via the identification of the light sources and the control of the light sources, can be dynamically adapted to changing legal, regulatory or physical requirements for a specific irradiation process when irradiating living beings with optical radiation in the medical, cosmetic and/or therapeutic sector.

The present invention also supports compliance with physiological and/or legal maximum doses of radiation by the evaluation unit checking and coordinating an overall actual radiation intensity of a plurality of light sources, for example.

Furthermore, in a special embodiment, the evaluation unit can be designed to control a plurality of light sources in combination with one another based on information stored on the identification unit of a light source. In a particularly preferred embodiment, the evaluation unit coordinates a plurality of light sources in order to maintain an overall irradiation intensity that is as constant as possible, regardless of any power drop in individual light sources. This can be done, for example, by compensating for a drop in the output of one light source with an additional output from other light sources.

In a special embodiment, the evaluation unit is designed to carry out an automatic first calibration of a light source on the basis of the information from the identification unit. This first calibration can include, for example, a test run with a light source start and/or an irradiation process in conjunction with other light sources. The automatic first calibration can also include the setting of maximum powers in order to meet legal or physiological requirements, for example, as described above. For example, the evaluation unit can reduce the output of a new light source in order to obtain a desired radiation intensity.

In a special embodiment, the evaluation unit is designed to monitor the light sources that have been identified once by means of information from the identification unit. The monitoring can include parameters such as line consumption, starting peak, operating life, operating hours, etc.

In a particular embodiment, physical locks are provided on the light source and/or on the light source holder, which make it impossible to insert and fix a light source in the irradiation device so that the identification unit does not come to rest in the detection region. Such physical locks can be key systems, grooves, bulges, flanges or other locks that can be easily conceived by a person skilled in the art, which, for example, make it impossible for a light source to be incorrectly inserted and/or rotated in the holder.

Another aspect of the present invention relates to a computer program product. The computer program product is designed to carry out a series of steps in an irradiation device. In particular, the computer program product is provided for operating an irradiation device. The computer program product is designed in a first step to activate a communication device to generate a detection region. A second step includes the detection of one or more identification units that contain information specific to the light sources. A further step comprises the evaluation and/or storage of the light source-specific information in an evaluation unit.

In a particular embodiment, the computer program product further comprises the step of controlling the communication device and/or an irradiation program and/or at least one irradiation parameter on the basis of the information specific to the light source.

In the context of the present invention, the computer program product can be part of the firmware of the irradiation device. In particular, the computer program product can be integrated in the control module of such a device.

In a further special embodiment, the computer program product is designed to carry out at least one start routine. According to the present invention, a start routine can consist, for example, in activating a transmitter/receiver, in particular a near-field transmitter/receiver, of the communication device in such a way that an identification unit located in the detection region is activated. By activating the communication unit, light source-specific information can be read from the identification unit and fed to an evaluation unit and/or stored. In a further special embodiment, the computer program product is designed to match the light source-specific information with a predefined database of light source-specific information. In this way, for example, the authenticity of a light source can be ensured. In this case, the light source-specific information can include, for example, a specific unique code that can be verified using a database or a corresponding decryption.

In this particular embodiment, the computer program product can prevent the use, ie the activation, of the light source in the irradiation device until this verification step has been completed. Furthermore, in a further special embodiment, the computer program product can prevent the light source from being activated if, for example, the operating parameters of the light source do not correspond to regulatory specifications and/or the operating parameters of the light source indicate a defect.

It is a particular advantage of the present invention that in such a case the computer program product is able to automatically contact a corresponding service provider and report the defective or impermissible operating parameters. In such a case, appropriate maintenance and/or the organization of an appropriate replacement can be initiated.

In a further special embodiment, the computer program product is designed to carry out a first calibration of the light sources that have been identified in the detection region. In this example, the computer program product can automatically recognize newly installed light sources and carry out a corresponding calibration. In such an example, all of the light sources provided in an irradiation device, which are designed in combination to impinge on a patient and/or object, could be kept in a specific operating state. For example, the computer program product can dim individual light sources while the power of others is increased. As a result, the computer program product can ensure that a constant radiant power generated over the entire group of light sources is maintained.

In medical and/or cosmetic equipment in particular, the safety of the use of radiation devices is particularly important. In this case, the computer program product can ensure that no unauthorized use of the light sources is possible. The computer program product can also control the detection or the activation of the communication device to the extent that this is coordinated with the operation of the irradiation device as a whole. In this particular embodiment, for example, the computer program product can be designed to coordinate the activation of the communication device with a switch-off of the irradiation device. This can prevent the communication device from having to carry out its detection in an electromagnetic field, which may be heavily distorted by the light source.

Without being bound to this theory, there may be disturbances in the electromagnetic field, at least in the field of electromagnetic detection based on radio frequencies, if, for example, high-power light sources generate a corresponding radiation field.

A further advantage of the present invention can be seen in the fact that, independently of country-specific specifications, irradiation devices can be provided which dynamically implement a permissible irradiation protocol in their output by recognizing the light sources used.

These light sources are recognized by the computer program product or an evaluation unit and the corresponding operating parameters can be reproduced automatically. The operating parameters can either be uploaded to the device dynamically, i.e. after the corresponding light sources have been detected via the identification means, or a predefined set of operating parameters can already be present in an irradiation device according to the invention and a comparison with the light source-specific information read from the identification means leads to the provision of the correct operating parameters. In the context of the present invention, the computer program product can be stored locally on the irradiation device or, alternatively or additionally, the computer program product can be executed from a central computing system on peripheral irradiation devices.

If the evaluation unit uses information from the identification unit to control the light source, safe and reliable operation of only suitable light sources in the irradiation device can be guaranteed.

### DESCRIPTION OF THE FIGURES

Exemplary embodiments of the invention are described with reference to the following figures.

FIG. 1: shows schematically an arrangement with a device according to the invention with a light source.

FIG. 2: shows schematically an arrangement with a further device according to the invention with a plurality of light sources.

FIG. 3: shows a schematic representation of an arrangement with an identification unit relative to the communication device and the possibility of rotation by an angle.

### EMBODIMENT OF THE INVENTION

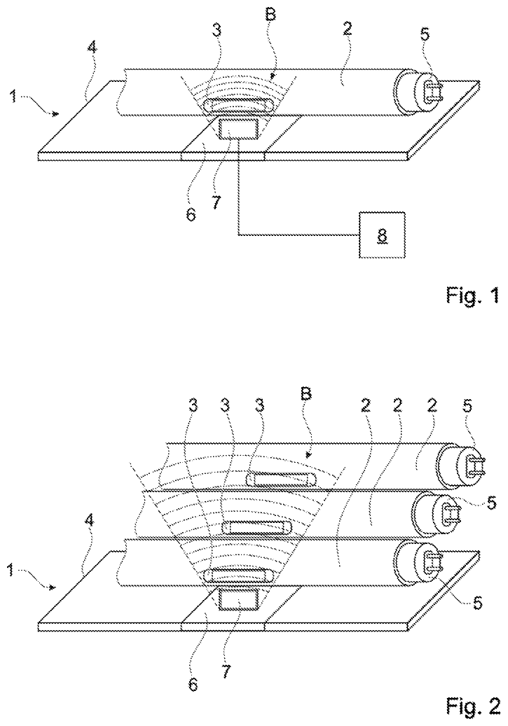

FIG. 1 shows schematically how an embodiment of an irradiation device 1 according to the invention is designed for subjecting an irradiation object to light. Such irradiation serves humans or other living beings for many medical or therapeutic purposes. FIG. 1 shows a light source 2, for example a lamp, which emits a special radiation in the visible or non-visible light spectrum. An identification unit 3, which is also referred to as an emitter, is fitted on or in the light source 2. The light source 2 is fastened to a reflective frame 4. This frame 4 is designed as a metal trough 4, which provides shielding and specifies the direction of the light for irradiation. The frame is preferably curved and designed for optimal irradiation. On the frame 4 there is a holder or light source holder 5 to which the light source 2 is fastened. The light source 2 is preferably snapped into the light source holder 5 by inserting it in a form-fitting manner.

The frame 4 has a region 6 in or on which a communication unit 7 is arranged. An evaluation or control unit 8 is connected to the communication unit 7.

A detection field B extends from the region 6, which can be referred to as a defined or predetermined region. The detection field B is generally not visible and is therefore indicated with dashed lines in the figure. The communication unit 7 is able to read the identification unit 3 by inductive coupling, for example.

Furthermore, it is also possible for the communication unit 7 to write information into the identification unit 3. A write/read process with low inductive coupling is preferred.

It has been shown that the materials have a decisive influence on the result of read and/or write cycles which take place in the vicinity of the communication unit 7. The region 6 is provided because interfering objects such as metal plates or materials such as aluminum or iron disturb the inductive coupling. The arrangement of a plastic plate already enables better inductive coupling. Milling out the frame 4 or the metal trough and introducing a non-conductive region 6, which preferably consists of plastic, is even better.

Without being bound by this theory, the electromagnetic field in the vicinity of metal surfaces may induce eddy currents that oppose the exciting magnetic flux. This is known as Lenz's law and leads to field weakening. Non-conductive materials do not lead to any significant field weakening.

Ferrite shielding is also beneficial, e.g. using a thin plate between the communication unit 7 and the metal frame 4.

It is particularly preferred if the communication unit 7 is arranged almost parallel to the identification unit 3, with a slight rotation being permissible. A direct arrangement one above the other of identification unit 3 and communication unit 7

The communication unit 7 provides an antenna which acts on the identification unit 3, also referred to as an RF identification unit, by means of inductive coupling and can read out the information stored in the passive identification unit 3. Writing information to the identification unit 3 is also possible.

Good results can be achieved in the HF range with high-frequency RFID technology at 13.56 MHz and inductive coupling in the near field. The MHz range is available worldwide. The size of the communication unit 7 must be matched to the identification unit 3. A very large HF antenna or communication unit 7 for simultaneous reading a plurality of identification units 3 cannot be expected to have any advantages due to the metallic environment.

In the UHF range, the approved channels K4: 865.7 MHz; K10: 866.9 MHz; K7: 866.3 MHz; or K14: 867.5 MHz can be used. The better the directional properties of the communication unit 7, the less power has to be fed in. In order to enable stable communication, the RF power must be adjusted accordingly. The transmission and reception properties are partly channel-dependent, despite the small frequency differences.

FIG. 2 shows a schematic representation of a further embodiment of the irradiation device 1 with a plurality of light sources 2. The light sources 2 are fastened to the reflective frame 4. Each light source 2 is attached to an associated holder or light source holder 5. Here, too, the light sources 2 are preferably snapped into place in the respective light source holder 5 by being inserted in a form-fitting manner.

The frame 4 has a region 6 in or on which a communication unit 7 is arranged. A control unit is connected to the communication unit 7, but is not shown.

The detection field B, which, in this case, covers several identification units 3, extends from the region 6. The communication unit 7 is capable of reading or writing to multiple identification units 3. This can preferably be realized with UHF units.

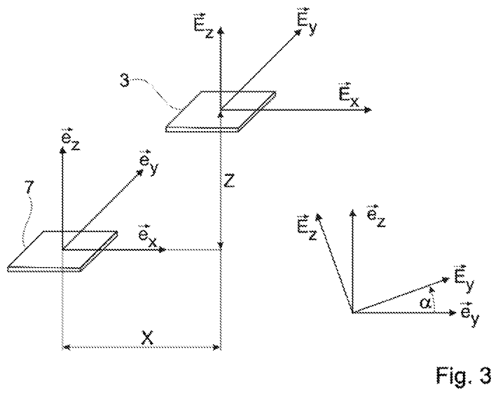

FIG. 3: shows a schematic representation of an arrangement with an identification unit 3 relative to the communication device 7 and the possibility of rotation by an angle α. However, this angle α should be as small as possible. The vectors of the identification unit 3 are shown with \vec{E} and those of the communication device 7 are shown with \vec{e}. The best results for reading and/or writing operations are obtained with a minimum distance x, z of the center points.

1. An irradiation device for irradiating an irradiation object with light, comprising:
at least one light source with an identification unit which contains light source-specific information;
a frame, in particular a reflective frame, with at least one light source holder, on which the at least one light source is arranged by form-fitting insertion into the light source holder;
a region which enables a detection field of a communication device;
wherein the identification unit is positioned on at least one light source in such a way that the identification unit is positioned within the detection field by form-fitting insertion into the light source holder.

2. The irradiation device of claim 1, wherein the detection field extends substantially perpendicularly from the frame.

3. The irradiation device of claim 1, wherein the frame is metallic and defines an irradiation direction.

4. The irradiation device of claim 1, wherein the region is designed as a recess in the frame, preferably as a milled out region.

5. The irradiation device of claim 1, wherein the region is non-metallic and preferably consists of a plastic.

6. The irradiation device of claim 1, wherein the identification unit contains a specific coding.

7. The irradiation device of claim 1, wherein the identification unit is an RF identification unit, from which information can be read.

8. The irradiation device of claim 1, wherein the identification unit is an RF identification unit, on which information can be written.

9. The irradiation device of claim 1, wherein the distance between the identification unit and the communication device is greater than the distance between the communication device and the region.

10. The irradiation device of claim 1, wherein the distance between the communication device and the frame is between 2 mm and 10 mm.

11. The irradiation device of claim 1, wherein a plurality of light sources are each provided with an identification unit and a communication device is assigned to each identification unit.

12. The irradiation device of claim 1, wherein a plurality of light sources are each provided with an identification unit and precisely one communication device is assigned to each of them.

13. The irradiation device of claim 1, wherein the communication device is connected to an evaluation unit.

14. The irradiation device of claim 13, wherein the evaluation unit uses information from the identification unit to control the light source.

15. A method for irradiating an irradiation object with light, comprising the steps:
providing at least one light source with an identification unit which contains light source-specific information;
positioning of the at least one light source in a frame, in particular a reflective frame, with at least one light source holder, by form-fitting insertion into the light source holder;
providing a detection field of a communication device in a defined region; and
positioning the identification unit on at least one light source in such a way that the identification unit is positioned within the detection field by form-fitting insertion into the light source holder.

16. A computer program product for operation in an irradiation device for irradiating an irradiation object with light, and which is designed for carrying out the following steps:
a. activating a communication device to generate a detection region;
b. detecting one or more identification unit(s) which contain light source-specific information;
c. evaluating and/or storing the light source-specific information in an evaluation unit.

17. A computer program product, further comprising the step:
a. controlling the communication device and/or an irradiation program and/or at least one irradiation parameter on the basis of the light source-specific information.

18. A light source for irradiating an irradiation object with light, comprising:
an identification unit which contains light source-specific information, the identification unit being positioned on the light source in such a way that the identification unit is positioned within a detection field by form-fitting insertion into a light source holder, which field is made possible by a region.
